(19) United States
(12) Patent Application Publication
(10) Pub. No.: US 2010/0112085 A1
(43) Pub. Date: May 6, 2010

(54) AGENT FOR STIMULATING LYMPHATIC DRAINAGE, METHOD FOR OBTAINMENT THE AGENT, AND METHOD FOR USING/ADMINISTERING THE AGENT

(76) Inventors: Mikhail Sergeevich Pikalov, Moscow (RU); Aleksandr Mikhailovich Pikalov, Moscow (RU)

Correspondence Address: Aleksandr Smushkovich, POB 140505, Brooklyn, NY, 11214, US

(21) Appl. No.: 12/450,407

(22) PCT Filed: Feb. 27, 2008
(86) PCT No.: PCT/RU2008/000104

§ 371 Date: Sep. 24, 2009

(30) Foreign Application Priority Data
May 17, 2007 (RU) 2007118217

(51) Int. Cl. A61K 33/00

(52) U.S. Cl. 424/600

(57) ABSTRACT

A lymphatic drainage stimulant is disclosed, which is produced by -providing a body of drinking water that meets predetermined sanitary requirements, and -treating the water body with a baro-membranous system. The treating preferably includes passing the water through a semi-permeable reverse-osmosis membrane made with a hole size 0.0001-0.005 μm, so that the water acquires the following parameters: a redox potential ranging from +200 to +343 mV, total mineralization ranging from 25 to 130 mg/L, and pH index ranging from 6.9 to 8.3. An optional method additionally includes saturating the water with macro- and microelements. The stimulant can be administered for medical treatment of patients with chronic skin diseases, non-oncological diseases of gastrointestinal tract, and chronic obstructive bronchitis. In such case, the stimulant is taken 30-40 minutes before and 2-2.5 hours after each meal, altogether 1.5-2.5 L a day, and the first dose is taken during the morning fast.

### CROSS-REFERENCE TO RELATED APPLICATIONS

This application is a U.S. national stage application of a PCT application PCT/RU2008/000104 filed on 27 Feb. 2008, published as WO/2008/143545, whose disclosure is incorporated herein in its entirety by reference, which PCT application claims priority of a Russian Federation application RU2007118217 filed on 17 May 2007.

### FIELD OF THE INVENTION

The invention relates to the chemical-pharmaceutical industry, namely to obtaining an agent (herein also called ‘stimulant’) capable of stimulating a lymphatic drainage including formation and transportation of the lymph.

### BACKGROUND OF THE INVENTION

Under conditions of total organism contamination with anthropogenic products the problem of science-based purification of an organism and above all of a habitat of cells has become today one of the main problems of health preservation of all the population of the planet.

Lympho-stimulation promotes elimination of metabolites, various xenobiotics out of tissues through the lymphatic system at their primary detoxication (deactivation) at the lymph node level, which contributes to detoxication of tissues, organs, and an organism as a whole.

Organism detoxication is performed by various methods; one of them is the balneal influence on organism tissues and organs, which is directed at invigoration of the organism internal environment with the help of baths, shower-bath and sauna (V. T. Olifirenko, Water-and-Thermal Cure, Moscow, Medicine, 1986, 288 p.), and all these hydrotherapeutic procedures may be accompanied by addition of aromatic, mineral, radio-active, and other agents.

Physical lymphatic stimulants (local irritants, massage, and curative gymnastics) are also widely used (R. T. Panchenkov and others, Lymphostimulation, Moscow., Medicine, 1986, 240 p.).

The disadvantage of well-known lympho-stimulation methods and preparations is that they have a local effect and moreover, the influence itself may produce an irritating effect on a patient.

### BRIEF DESCRIPTION OF THE INVENTION

The proposed invention allows achieving the improvement of natural drainage and the detoxication function of an organism.

The primary objects of this invention are to create a lymphatic drainage stimulant on the basis of a natural substance, namely processed drinking water; to provide a method for obtaining such stimulant; and to provide a method for using/administering such stimulant. Other objects may be recognized by those skilled in the art upon learning the present disclosure, and therefore encompassed in the scope of this invention.

A lymphatic drainage stimulant is herein proposed, which is composed of drinking water subjected to baro-membranous treatment and having redox potential from +200 to +343 mV, total mineralization from 25 to 130 mg/1 and pH index from 6.9 to 8.3, its daily consumption dose makes up 1.5 to 2.5 L.

The stimulant may be used in complex therapy for patients with chronic skin diseases, non-oncological diseases of gastrointestinal tract and chronic obstructive bronchitis.

A method of lymphatic drainage stimulant obtainment is herein proposed, according to which a body of water is first brought to the state of drinking water and then is treated in a baro-membranous processing device, where the water is passed through membranes made of a semi-permeable material with a hole size of 0.0001-0.005 μm.

The so purified water has the following characteristics:

| | |
|---|---|
| redox potential | from +200 to +343 mV |
| total mineralisation | from 25 to 130 mg/l |
| pH index | from 6.9 to 8.3. |

Furthermore, the purified water may be subjected to an additional treatment by saturating it with macro- and microelements.

Another method for lymphatic drainage stimulation is herein disclosed that can be used/administered for medical treatment of patients with chronic skin diseases, non-oncological diseases of gastrointestinal tract and chronic obstructive bronchitis with the help of the above-described inventive stimulant, and the water is taken 30-40 minutes before and 2-2.5 hours after each food intake (meal), altogether 1.5-2.5 L a day, the first dose is taken on an empty stomach.

### BRIEF DESCRIPTION OF THE DRAWINGS

FIG. 1 shows a schematic diagram of conventionally known lymphatic drainage of tissues.

FIG. 2 is a graph illustrating a complete evacuation time (in minutes) of a lymphotropic dye depending on the group of animals (main group and check group), according to an embodiment of the present invention.

FIG. 3 is a graph illustrating a complete evacuation time (in percents of the check group) of a lymphotropic dye depending on the group of animals (main group and check group), according to an embodiment of the present invention.

FIG. 4 is a graph illustrating a volumetric lymph flow from the thoracic duct (1 hr later), depending on the group of animals (main group and check group), according to an embodiment of the present invention.

FIG. 5 is a graph illustrating a volumetric lymph flow from the thoracic duct (2 hrs later), depending on the group of animals (main group and check group), according to an embodiment of the present invention.

### DETAIL DESCRIPTION OF PREFERRED EMBODIMENTS OF THE INVENTION

While the invention may be susceptible to embodiment in different forms, there are described in detail herein, specific embodiments of the present invention, with the understanding that the present disclosure is to be considered an exemplification of the principles of the invention, and is not intended to limit the invention to that as exemplified herein.

The invention is illustrated with the following examples of obtainment and use of the claimed water stimulant in experiments on animals and volunteers.

The drinking cold tap water, which has been previously purified by municipal services and meets the requirements of sanitary standard SanPiN 2.1.4.1074-01 “Drinking Water” is additionally purified (for example, with the help of a domestic reverse-osmosis filter “Atoll”) before consumption. As widely known, a reverse-osmosis filter system is a particular type of the abovementioned baro-membranous systems.

“Atoll” is a reverse-osmosis system that typically serves for additional purification of water in common household conditions in order to use it subsequently for drinking, cooking, and other conventional purposes. “Atoll” removes up to 99.9% of all impurities contained in water and prevents formation of scale in heaters. At the same time, the water while going through the filter gets enriched in oxygen that additionally gives it a pleasant fresh taste. The purification method of reverse osmosis, applied in these systems, does not imply using of chemicals and is accomplished by passing the water under pressure through a special membrane.

The “Atoll” reverse-osmosis systems are produced in Russia (according to specifications TU3697-009-18261557-03) and in the USA (NSF/ANSI Standard 058). The water is passed through membranes made of a semi-permeable material with a hole size 0.0001-0.005 μm; the purified water has the following characteristics:

| | |
|---|---|
| redox potential | from +200 to +343 mV |
| total mineralisation | from 25 to 130 mg/l |
| pH index | from 6.9 to 8.3. |

Furthermore, the purified water may be subjected to additional treatment by saturating it with macro- and microelements (calcium, magnesium, sodium, sulfur, vanadium, carbon, oxygen; iron, iodine, copper, cobalt, zinc, fluorine, selenium, silicon, silver etc.).

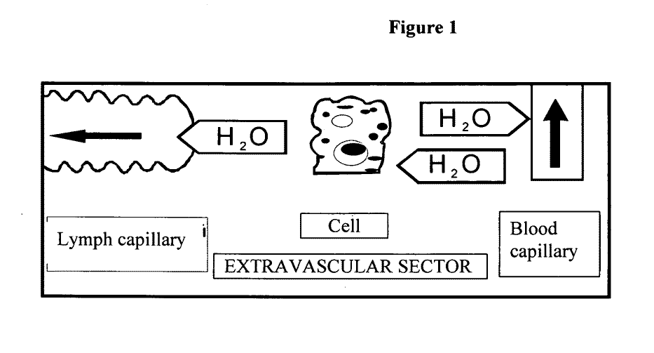

As well known, lymphatic drainage of tissues (whose schematic diagram is shown on FIG. 1) is the main way of organism cell habitat purification from metabolic waste excreted by them (which waste comes from the blood), and also from toxic substances formed in the tissues.

The experiments have been conducted on white common mice and Chinchilla rabbits.

Experiments on Mice.

The animals were kept in a special, separate premise with a room temperature and daylight. The mice had a free access to water and food. All of them were kept in the same conditions before the experiment. Then the animals were divided into groups (10 individuals in each). One group was given the claimed stimulant water for drinking. The other group of laboratory mice in a series of check experiments were drinking settled tap water during the whole period of time.

After the expiration of the designed period of drinking of the claimed stimulant water 0.1% sodium pentobarbital solution was introduced intraperitoneally to the animals on the basis of 0.8 ml per 100 G of the body weight.

After that a midline laparotomy was performed. A part of a small intestine with a frill were extracted, placed on a special heated table and sprinkled with isotonic solution. The sample table and isotonic solution temperature was maintained at a physiological level.

A mark (Evance blue), which could be eliminated from the place of introduction only through the lymphatic system, was introduced with a syringe in the amount of 0.02 ml in a standard place (ileocecal angle) into a cellular tissue situated along the vessels. The time of mark introduction and excretion was regularly fixed. The time of the dye evacuation into the lymphatic system was estimated, which defined a speed of extravascular humoral transportation and lymphatic drainage of tissues.

The data obtained were subjected to statistical analysis. The results obtained were processed with the help of a parametric (Student's test) and a nonparametric (Man-Witney criterion and Dann criterion) methods of assessment. The results with P>95% were considered to be reliable.

The claimed water influence on the lymphatic drainage when used for 5-7 days:

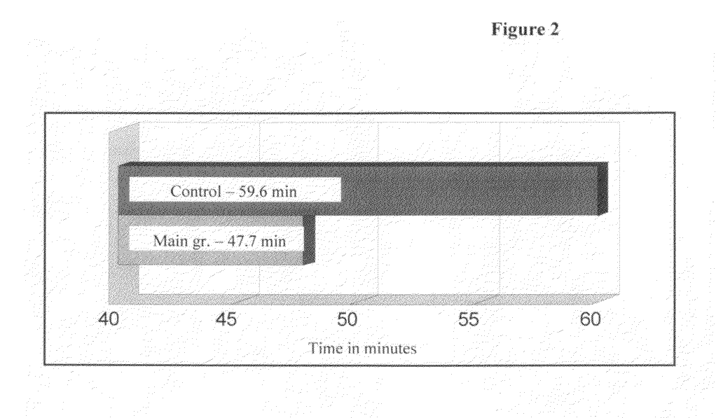

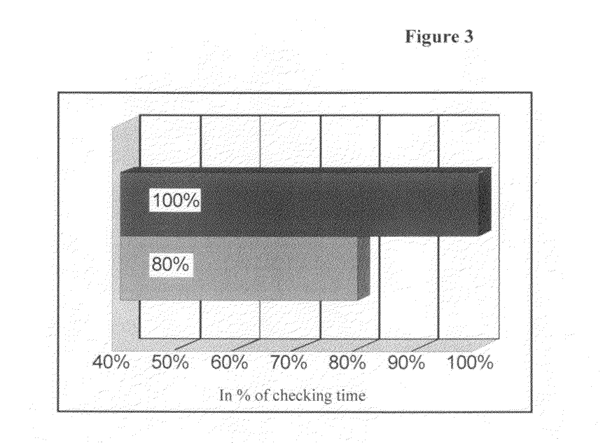

The data of ten experiments in the main group and ten experiments in the check group. The complete evacuation time of a lymphotropic dye is plotted on FIG. 2, (and it is plotted on FIG. 3 in percentage): Main group—47.7 min. Check group—59.6 min. The difference is of high statistical reliability.

The experiments on mice have ascertained that weekly ingestion of the claimed water stimulant accelerates lymphatic drainage by 20%-30%.

The claimed water influence on the lymphatic drainage when used for 14-17 days:

The data of ten experiments in the main group and ten experiments in the check group have shown identical results with the data obtained when using the water during 5-7 days.

Experiments on Chinchilla Rabbits.

The experiment was conducted on six Chinchilla rabbit males weighing 3-3.5 KG.

The claimed water had been slowly evaporated till the initial quantity reduced three times. Then a concentrated solution of NaCl was added to the remained solution up to the initial volume with attainment of its isotonicity.

The following stages were common for all the experiments:

1—narcotisation of the animals by introducing sodium pentobarbital into the marginal auricular vein in the quantity of 20 mg/kg of the anumal's weight;

2—tracheotomy with connection of an artificial pulmonary ventilation apparatus;

3—thoracotomy;

4—thoracic (lymphatic) duct catheterization by the method of A. A. Kornienko and co-authors to determine a lymph efflux rate;

5—femoral vein catheterization to fill up an amount of liquid lost with the lymph, to introduce the examined solution, which contained the claimed water stimulant ingredients.

After the thoracic duct draining, a lymph outflow rate was determined by collecting the lymph into a graduated marked test-tube during two hours, every 20 minutes measuring the quantity exuded. To maintain a water-salt balance, every 40 minutes an isotonic solution of NaCl was injected intravenously in the amount equal to the lost lymph quantity.

The so measured volumetric lymph flow from the thoracic duct (ml/min) is shown below:

Checking of 6 experiments

| | |
|---|---|
| 1-st hour | 0.065 ± 0.004 |
| 2-nd hour | 0.067 ± 0.004 |

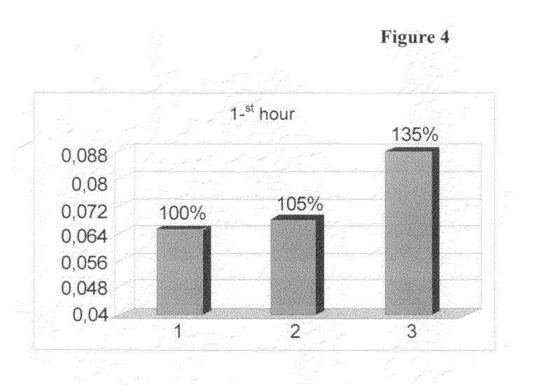

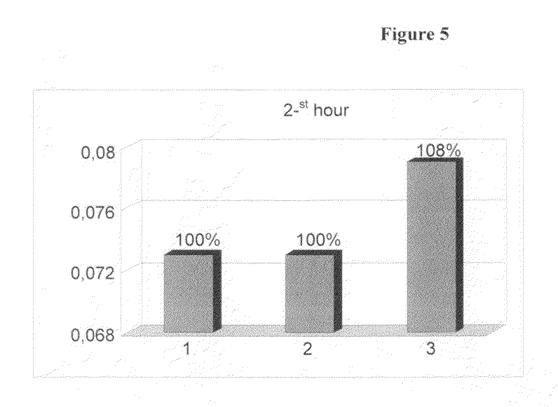

One hour after the thoracic duct cutting, an adequate quantity of solution containing the claimed water ingredients was introduced instead of isotonic solution. The data obtained are reflected in FIGS. 4 and 5. The effect is highly reliable.

(M ± m; n = 6)

| | | After introduction | |
|---|---|---|---|
| | | Solution of | |
| | | NaCl | solution with the declared |
| Before introduction | | (checking) | water ingredients |
| 1-st hour | 0.065 ± 0.004 | 0.068 ± 0.004 | 0.088 ± 0.005 |
| 2-nd hour | 0.073 ± 0.004 | 0.073 ± 0.004 | 0.079 ± 0.004 |

The experiment data show that the claimed water characteristics stimulate the lymph formation.

An ability of the claimed water stimulant to accelerate the lymphatic drainage of tissues was studied on animals.

Administering the claimed water stimulant was included into a complex therapy of 84 patients (that was conducted in the city of Pushchino, Russian Federation). Three series of observations were performed:

1. Chronic locomotor system diseases—28 patients;

2. Skin diseases—28 patientsB;

3. Gastrointestinal tract pathology—28 patients.

6 groups of 14 patients were formed: three check groups and three main groups. All the groups were comparable in sex, age, and time of chronic inflammatory disease.

The first three groups were administered usual boiled water, the rest three groups took the claimed water stimulant 30-40 minutes before and 2-2.5 hours after each food intake, altogether 1.5-2.5 liters a day, and the first intake was on an empty stomach. The water stimulant was additionally saturated with macro- and microelements, but the total mineralization did not exceed the limits of 25-130 mg/L.

The medical treatment efficiency was estimated by clinical manifestations, biochemical blood analysis results, ultrasonic scanning data before and after the treatment.

The claimed water stimulant included into a complex therapy of chronic locomotor system diseases, skin diseases, and gastrointestinal tract pathology has shown an expressed curative effect on patients in comparison with the check groups.

Thus, the above-described water stimulant has a unique stimulating effect on the organism tissue lymphatic drainage, including the lymph formation and transportation.

The revealed effect makes it possible to use the claimed water stimulant for endo-ecological rehabilitation of an organism at the cellular-organism level owing to 20-30% acceleration of the lymphatic drainage of tissues.

Explanations to FIGS. 4 and 5:

Column 1—checking before the introduction;

Column 2—the introduction of NaCl isotonic solution (check 2);

Column 3—the introduction of isotonic solution with the claimed water ingredients.

1. A lymphatic drainage stimulant produced through a method, comprising the steps of
providing a body of drinking water that meets predetermined sanitary requirements;
additionally treating said water body with a baro-membranous system, said treating includes passing said water body through a semi-permeable membrane so that said water body acquiring the following parameters:
a redox potential ranging from +200 to +343 mV,
total mineralization ranging from 25 to 130 mg/L, and
pH index ranging from 6.9 to 8.3.

2. The stimulant according to claim 1, wherein said water body is additionally saturated with macro- and microelements.

3. The stimulant according to claim 1, wherein said predetermined membrane is made of a semi-permeable material with a hole size of 0.0001-0.005 μm.

4. A method for obtaining a lymphatic drainage stimulant, said method comprises the steps of:
providing a body of drinking water that meets predetermined sanitary requirements;
additionally treating said water body with a baro-membranous system, said treating includes passing said water body through a semi-permeable membrane so that said water body acquiring the following parameters:
a redox potential ranging from +200 to +343 mV,
total mineralization ranging from 25 to 130 mg/L, and
pH index ranging from 6.9 to 8.3, thereby obtaining said stimulant.

5. The method according to claim 4, wherein said water body is additionally saturated with macro- and microelements.

6. The method according to claim 4, wherein said semi-permeable membrane is made of a semi-permeable material with a hole size 0.0001-0.005 μm.

7. The stimulant according to claim 1, wherein said baro-membranous system is represented by a reverse-osmosis filter system.

8. The method according to claim 4, wherein said baro-membranous system is represented by a reverse-osmosis filter system.

9. A method for administering the lymphatic drainage stimulant produced according to claim 1 to patients, said method is provided for medical treatment of said patients with chronic skin diseases, non-oncological diseases of gastrointestinal tract, and chronic obstructive bronchitis; said method comprises the steps of:
taking a first dose of the stimulant 30-40 minutes before each meal, and
taking a second dose of the stimulant 2-2.5 hours after each meal;
wherein a total daily dose, consisting of said first and said second doses, taken per day, ranges from 1.5 to 2.5 L, and said first dose is taken before the first meal on empty stomach.
